(19) United States
(12) Patent Application Publication
(10) Pub. No.: US 2019/0134247 A1
(43) Pub. Date: May 9, 2019

(54) FAN WITH AIR FRESHENING DISPENSER

(71) Applicants: KINGSLEY OLIGIE, COLUMBIA, MD (US); LINDA GILMORE OLIGIE, COLUMBIA, MD (US)

(72) Inventors: KINGSLEY OLIGIE, COLUMBIA, MD (US); LINDA GILMORE OLIGIE, COLUMBIA, MD (US)

(21) Appl. No.: 15/805,379

(22) Filed: Nov. 7, 2017

(51) Int. Cl. A61L 9/12; F04D 29/00; F04D 29/70; F04D 27/00; F04D 25/06; F04D 25/10; F04D 29/32

(52) U.S. Cl. CPC A61L 9/122; F04D 29/002; F04D 29/703; F04D 27/008; A61L 2209/133; F04D 25/105; F04D 29/325; A61L 2209/111; F04D 25/06

(57) ABSTRACT

The fan with air freshening dispenser comprises a fan operated by an electrical motor, which dispenses a scent from an air freshener located at the front center of the fan. The air freshener is held within an air freshener compartment, which may be opened to replenish the air freshener. The fan comprises a stand suitable for use on a support surface, such as a desk or table, or on the floor. The fan with air freshening dispenser comprises an electronics unit, which provides clock, alarm, and timer functions. A remote control allows the fan with air freshening dispenser to be operated from a distance.

### CROSS REFERENCES TO RELATED APPLICATIONS

Not Applicable

### STATEMENT REGARDING FEDERALLY SPONSORED RESEARCH Not Applicable

### REFERENCE TO APPENDIX

Not Applicable

### BACKGROUND OF THE INVENTION

### Field of the Invention

The present invention relates to the field of environmental control, more specifically, a fan with an air freshening dispenser.

### SUMMARY OF INVENTION

The fan with air freshening dispenser comprises a fan operated by an electrical motor, which dispenses a scent from an air freshener located at the front center of the fan. The air freshener is held within an air freshener compartment, which may be opened to replenish the air freshener. The fan comprises a stand suitable for use on a support surface, such as a desk or table, or on the floor. The fan with air freshening dispenser comprises an electronics unit, which provides clock, alarm, and timer functions. A remote control allows the fan with air freshening dispenser to be operated from a distance.

An object of the invention is to provide a fan that dispenses a scent from an air freshener.

Another object of the invention is to provide the fan with an electronics unit that comprises clock, alarm, and timer functions.

A further object of the invention is to provide a remote control for the fan.

These together with additional objects, features and advantages of the fan with air freshening dispenser will be readily apparent to those of ordinary skill in the art upon reading the following detailed description of the presently preferred, but nonetheless illustrative, embodiments when taken in conjunction with the accompanying drawings.

In this respect, before explaining the current embodiments of the fan with air freshening dispenser in detail, it is to be understood that the fan with air freshening dispenser is not limited in its applications to the details of construction and arrangements of the components set forth in the following description or illustration. Those skilled in the art will appreciate that the concept of this disclosure may be readily utilized as a basis for the design of other structures, methods, and systems for carrying out the several purposes of the fan with air freshening dispenser.

It is therefore important that the claims be regarded as including such equivalent construction insofar as they do not depart from the spirit and scope of the fan with air freshening dispenser. It is also to be understood that the phraseology and terminology employed herein are for purposes of description and should not be regarded as limiting.

### BRIEF DESCRIPTION OF DRAWINGS

The accompanying drawings, which are included to provide a further understanding of the invention are incorporated in and constitute a part of this specification, illustrate an embodiment of the invention and together with the description serve to explain the principles of the invention. They are meant to be exemplary illustrations provided to enable persons skilled in the art to practice the disclosure and are not intended to limit the scope of the appended claims.

FIG. 1 is a perspective view of an embodiment of the disclosure.

FIG. 2 is a front view of an embodiment of the disclosure.

FIG. 3 is a side view of an embodiment of the disclosure.

FIG. 4 is a detail view of an embodiment of the disclosure showing the air freshener compartment.

### DETAILED DESCRIPTION OF THE EMBODIMENT

The following detailed description is merely exemplary in nature and is not intended to limit the described embodiments of the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to practice the disclosure and are not intended to limit the scope of the appended claims. Furthermore, there is no intention to be bound by any expressed or implied theory presented in the preceding technical field, background, brief summary or the following detailed description. As used herein, the word “or” is intended to be inclusive.

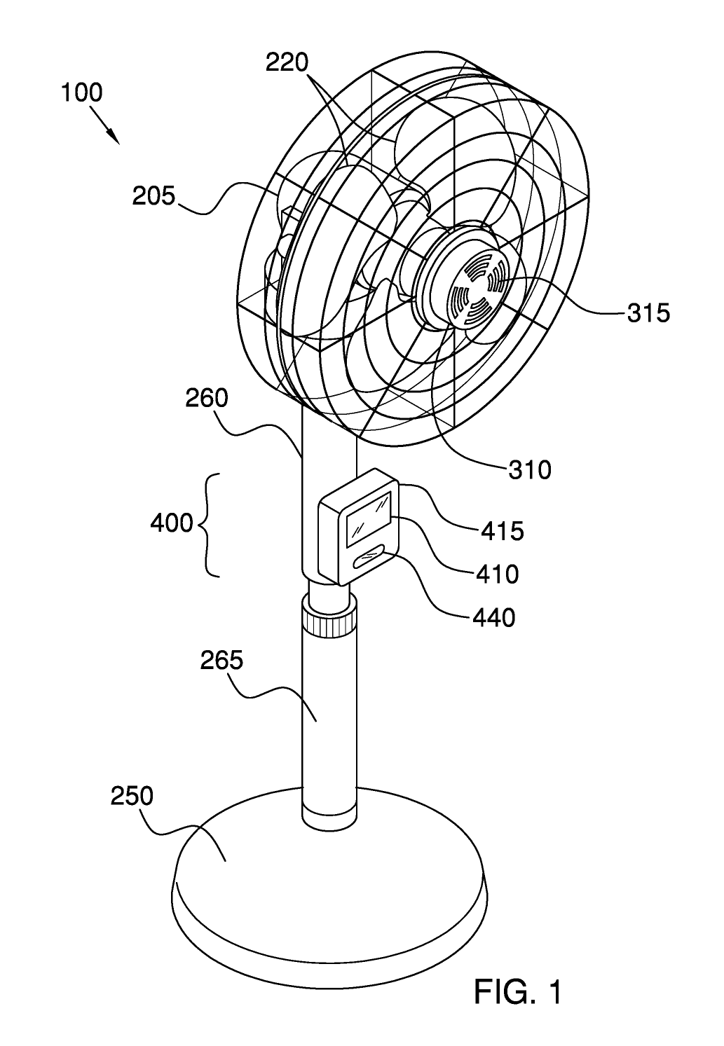

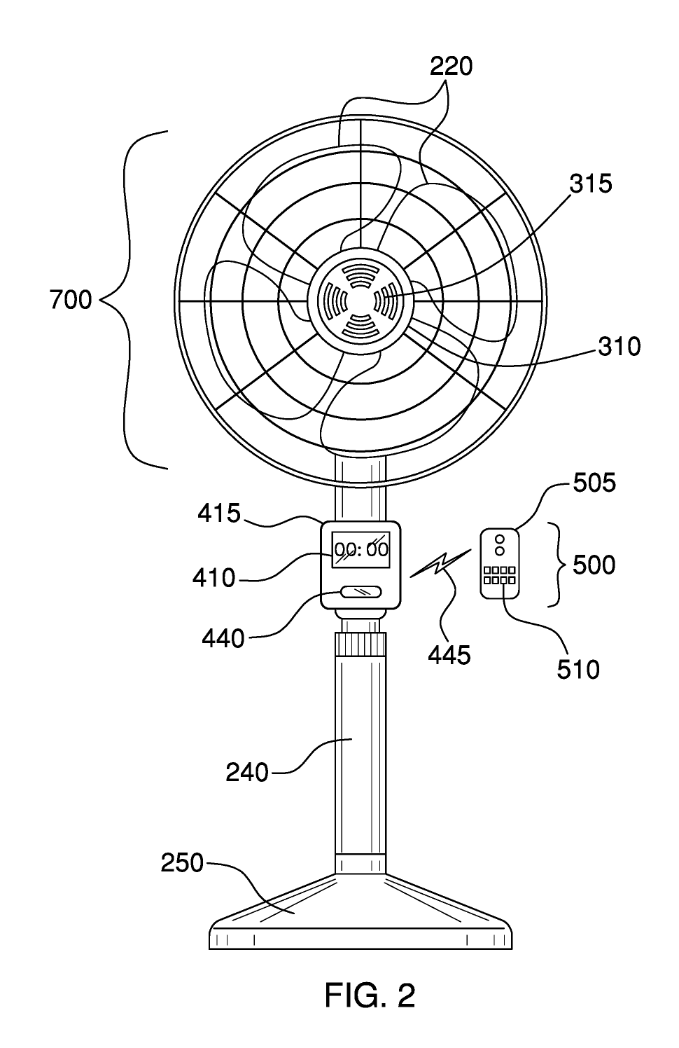

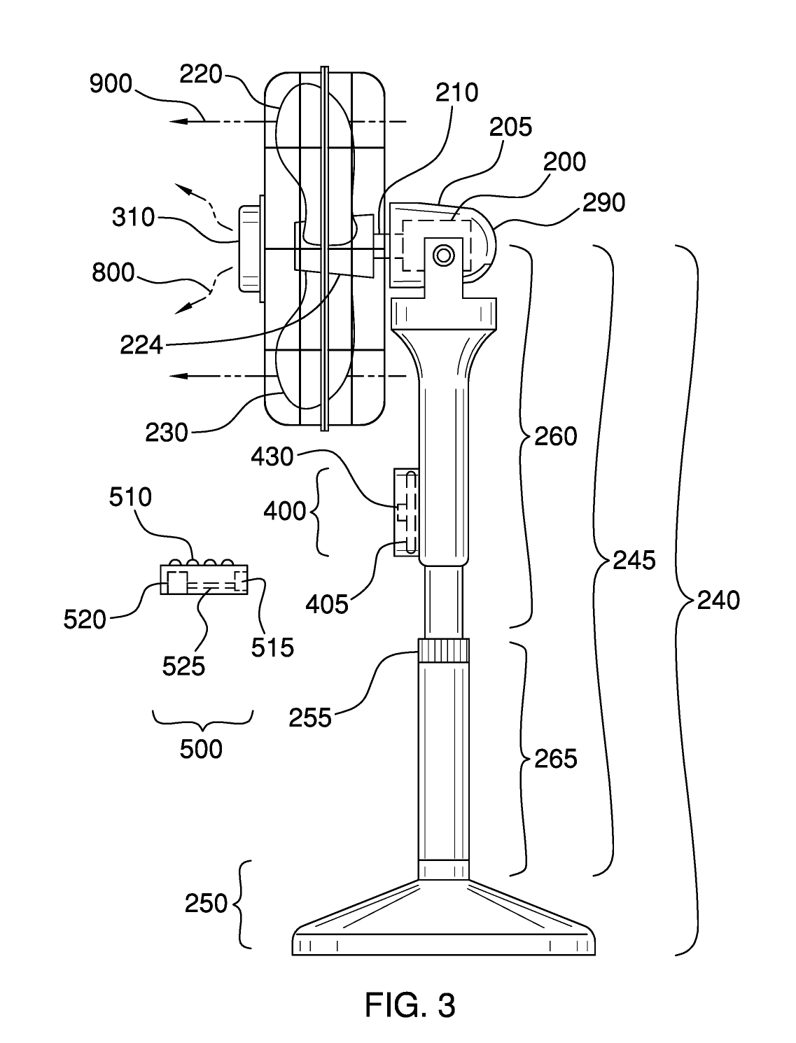

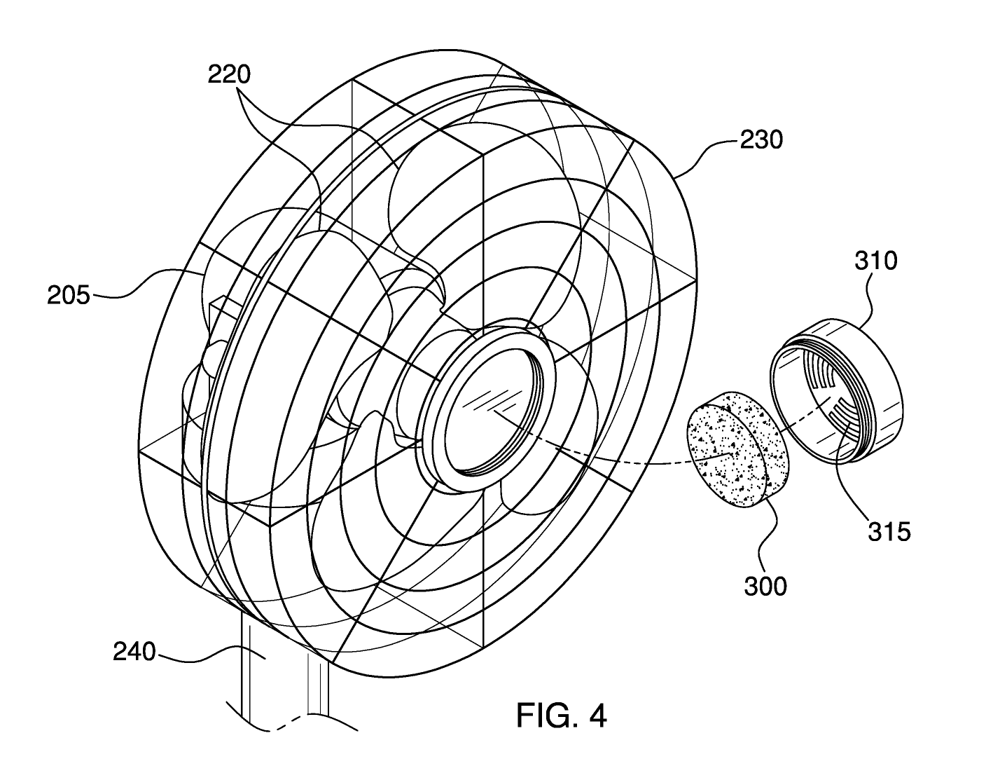

Detailed reference will now be made to a first potential embodiment of the disclosure, which is illustrated in FIGS. 1 through 4.

The fan with air freshening dispenser 100 (hereinafter invention) comprises an air freshener compartment 310, an electronics module 400, a remote control 500, a motor housing 205, a motor 200, plurality of fan blades 220, a blade cover 230, and a stand 240. The motor housing 205, the motor 200, the plurality of fan blades 220, and the blade cover 230 may comprise a fan 700. The air freshener compartment 310 located at the front of the fan 700 may introduce a scent 800 into an air flow 900 created by the fan 700. The electronics module 400 may comprise a clock (not illustrated in the figures), an alarm (not illustrated in the figures), and a timer (not illustrated in the figures). The clock, the alarm, the timer, and motion of the plurality of fan blades 220 may be controlled by the remote control 500.

The air freshener compartment 310 may be a circular container at the front center of the blade cover 230. The air freshener compartment 310 may hold an air freshener 300. The air freshener 300 may be a solid or a liquid. The solid form is illustrated in the figures. The air freshener compartment 310 may comprise a plurality of vents 315 to allow the scent 800 to escape from the air freshener compartment 310. The air freshener compartment 310 may open to allow replacement or refilling of the air freshener 300.

The electronics module 400 may comprise a housing 415, control circuitry 405, a sound transducer 430, and a display 410 mounted to the front of the stand 240. The control circuitry 405 may comprise the clock, the alarm, and the timer. The clock may track the time of day which the display 410 may show on the display 410. The display 410 may be visible on the front side of the electronics module 400. The alarm may be set to produce an alert noise using the sound transducer 430 at a specific time of day. The timer may be used to limit the duration of operation of the motor 200.

The electronics module 400 may further comprise a sensor 440 for receiving a control signal 445 sent by the remote control 500.

The remote control 500 may be a hand-held, battery-powered device which controls the operation of the invention 100 from a distance. The remote control 500 may comprise a remote control enclosure 505, a plurality of controls 510, a transmitter 515, remote control circuitry 525, and one or more batteries 520. The remote control enclosure 505 may be a container, which houses the remote control circuitry 525, the transmitter 515, the one or more batteries 520, and the plurality of controls 510. The plurality of controls 510 may be accessible for activation on the front side of the remote control enclosure 505. The remote control enclosure 505 may provide a battery door (not illustrated in the figures) to allow access to the one or more batteries 520.

The plurality of controls 510 may activate specific features of the invention 100. Activating one of the plurality of controls 510 may cause the remote control circuitry 525 to send the control signal 445 from the transmitter 515 to the sensor 440 on the electronics module 400. The control signal 445 may be encoded or modulated to indicate which of the plurality of controls 510 was activated.

In some embodiments, the plurality of controls 510 may be pushbuttons.

As non-limiting examples, activation of one or more of the plurality of controls 510 on the remote control 500 may cause the motor 200 to start, stop, or change speed, may start or stop the timer, may set, clear, or silence the alarm, or may change the clock time showing on the display 410.

The motor housing 205 may be a cowling to cover the motor 200. The motor housing 205 may prevent contact with moving parts or electrically energized circuits of the motor 200. The motor housing 205 may be pivotally coupled to the stand 240 on each side of the motor housing 205 to allow adjusting of the up/down angle of the fan.

In some embodiments, the rear of the motor housing 205 may comprise a manual speed control 290. The manual speed control 290 may allow the fan 700 to be turned on or off and may allow selection of a fan speed by turning the manual speed control 290. The manual speed control 290 may provide an input signal (not illustrated in the figures) to the control circuitry 405 in the electronics module 400 that allows the control circuitry 405 to combine input from the manual speed control 290 and input from the remote control 500 into a final determination of the activation state and speed of the fan 700.

The motor 200 may be an electrical motor. The motor 200 may cause rotation of a motor shaft 210 when the motor 200 is electrically energized. The motor 200 may be electrically energized from a power source. As non-limiting examples, the power source may comprise a power cord (not illustrated in the figures) plugged into a wall outlet or internal batteries (not illustrated in the figures) within the invention 100. The electronics module 400 may control the electrical energy reaching the motor 200.

The motor shaft 210 may be coupled to a central hub 224. Rotation of the motor shaft 210 may cause rotation of the plurality of fan blades 220 via the central hub 224.

In some embodiments, the motor 200 may cause oscillatory side-to-side motion of the fan 700 relative to the stand 240 through gearing and a crank mechanism (not illustrated in the figures).

The plurality of fan blades 220 comprise 2 to 5 paddles that are coupled to the central hub 224 and are inclined from being co-planar with each other. The plurality of fan blades 220 are all inclined in the same direction and by the same angle of inclination with respect to the central hub 224. When the motor 200 causes rotation of the central hub 224, the plurality of fan blades 220 work co-operatively with each other to push air through the plurality of fan blades 220 in the same direction.

The blade cover 230 is a protective cover over the plurality of fan blades 220. The blade cover 230 may be a mesh or grill that allows the air flow 900 through while preventing an object from touching the plurality of fan blades 220. The blade cover 230 surrounds the plurality of fan blades 220 and couples to the motor housing 205.

The stand 240 may be an upright support for the fan 700. The stand 240 may elevate the fan 700 above a support surface (not illustrated in the figures) or above the floor (not illustrated in the figures). As non-limiting examples, the support surface may be a table, counter, desk, shelf or other horizontal surface. The stand 240 may comprise an upright armature 245 and a base 250. The base 250 may rest on the support surface to provide a stable platform for the invention 100. The top of the base 250 may be coupled to the lower end of the upright armature 245. The upper end of the upright armature 245 may be pivotally coupled to the motor housing 205. The electronics module 400 may be coupled to the front of the upright armature 245 below the blade cover 230.

In some embodiments, the upright armature 245 may comprise a height adjustment 255. The height adjustment 255 may allow an upper section 260 of the upright armature 245 to slide relative to a lower section 265 of the upright armature 245 when the height adjustment 255 has been disengaged. The height adjustment 255 may allow the overall height of the fan 700 above the support surface or above the floor to be changed. Once the fan 700 has been moved to a desired height, the height adjustment 255 may be engaged to hold that height. As a non-limiting example, the height adjustment 255 may comprise a twist-lock ring that locks or releases a telescopic joining of the upper section 260 of the upright armature 245 and the lower section 265 of the upright armature 245.

Unless otherwise stated, the words “up”, “down”, “top”, “bottom”, “upper”, and “lower” should be interpreted within a gravitational framework. “Down” is the direction that gravity would pull an object. “Up” is the opposite of “down”. “Bottom” is the part of an object that is down farther than any other part of the object. “Top” is the part of an object that is up farther than any other part of the object. “Upper” refers to top and “lower” refers to the bottom. As a non-limiting example, the upper end of a vertical shaft is the top end of the vertical shaft.

Throughout this document the terms “battery”, “battery pack”, and “batteries” may be used interchangeably to refer to one or more wet or dry cells or batteries of cells in which chemical energy is converted into electricity and used as a source of DC power. References to recharging or replacing batteries may be construed to mean recharging or replacing individual cells, individual batteries of cells, or a package of multiple battery cells as is appropriate for any given battery technology that may be used.

As used in this disclosure, a “blade” is a term that is used to describe a wide and flat structure, or portion of a larger structure such as a propeller, or the cutting edge of a tool.

As used in this disclosure, a “clock” is an instrument that measures the passage of time. Clocks are often synchronized to a reference “time” that allows for communities to coordinate the scheduling of activities.

As used herein, the words “control” or “controls” are intended to include any device which can cause the completion or interruption of an electrical circuit; non-limiting examples of controls include toggle switches, rocker switches, push button switches, rotary switches, electromechanical relays, solid state relays, touch sensitive interfaces and combinations thereof whether they are normally open, normally closed, momentary contact, latching contact, single pole, multi-pole, single throw, or multi-throw.

As used herein, the words “couple”, “couples”, “coupled” or “coupling”, mean connected, either directly or indirectly and does not necessarily imply a mechanical connection.

As used in this disclosure, a “crank” is a handle or an arm that is attached perpendicularly to the axis of rotation of a shaft and that is used for transmitting rotary motion to the shaft.

As used herein, the word “desired” refers to a specific value within a range of supported values. A “desired” value indicates that a range of values is enabled by the invention and that a user of the invention may select a specific value within the supported range of values based upon their own personal preference. As a non-limiting example, for a fan that supports operational speed settings of low, medium, or high, a user may selected a desired fan speed, meaning that the user may select low, medium, or high speed based upon their needs and preferences at the time of the selection.

As used in this disclosure, a “display” is a surface upon which is presented an image, potentially including, but not limited to, graphic images and text, that is interpretable by an individual viewing the image. When used as a verb, “display” means to present such an image.

As used in this disclosure, a “fan” is a mechanical device with rotating blades that is used to create a flow or current of air.

As used herein, “front” means 1) the side of an object that is closest to a forward direction of travel under normal use of the object or 2) the side or part of an object that normally presents itself to view or that is normally used first. “Rear” or “back” is the side opposite the front.

As used herein, the term “height adjustment” refers to a mechanism that allows the overall height of an armature or stanchion to change by releasing a locking mechanism, sliding a first armature into or out of a second armature, and re-engaging the locking mechanism. As a non-limiting example, the locking mechanism may comprise a plurality of holes in the first armature and a plurality of holes in the second armature with a pin passing through the holes when they are in alignment. As a further non-limiting example, the locating mechanism may comprise a spring loaded button on an inside armature that pops through one of a plurality of holes in an outside armature and which can be pressed into the hole to release the locking mechanism. If the height adjustment comprises more than two armatures it may be known as a telescoping height adjustment.

As used in this disclosure, “horizontal” is a directional term that refers to a direction that is perpendicular to the local force of gravity. Unless specifically noted in this disclosure, the horizontal direction is always perpendicular to the vertical direction.

As used in this disclosure, a “housing” is a rigid casing that encloses and protects one or more devices.

As used herein, “inclined in the same direction and by the same angle of inclination” shall mean that as the central hub of the fan is rotated to bring a fan blade to a specific rotational position, the fan blade in the specific rotational position is indistinguishable from other fan blades when the other fan blades are in the specific rotational position.

As used herein, “oscillatory” or “vibratory” refer to a rhythmic or repetitious motion.

As used in this disclosure, a “sensor” is a device that receives and responds in a predetermined way to a signal or stimulus.

As used in this disclosure, the term “shaft” is used to describe a rigid cylinder that is often used as the handle of a tool or implement. The definition of shaft explicitly includes solid shafts or shafts that comprise a hollow passage through the shaft along the center axis of the shaft cylinder, whether the shaft has one or more sealed ends or not.

As used in this disclosure, a “transducer” is a device that converts a physical quantity, such as pressure or brightness into an electrical signal or a device that converts an electrical signal into a physical quantity.

With respect to the above description, it is to be realized that the optimum dimensional relationship for the various components of the invention described above and in FIGS. 1 through 4, include variations in size, materials, shape, form, function, and manner of operation, assembly and use, are deemed readily apparent and obvious to one skilled in the art, and all equivalent relationships to those illustrated in the drawings and described in the specification are intended to be encompassed by the invention.

It shall be noted that those skilled in the art will readily recognize numerous adaptations and modifications which can be made to the various embodiments of the present invention which will result in an improved invention, yet all of which will fall within the spirit and scope of the present invention as defined in the following claims. Accordingly, the invention is to be limited only by the scope of the following claims and their equivalents.

What is claimed is:

1. A fan with air freshening dispenser comprising:
an air freshener compartment, an electronics module, a remote control, a motor housing, a motor, plurality of fan blades, a blade cover, and a stand;
wherein the motor housing, the motor, the plurality of fan blades, and the blade cover comprises a fan;
wherein the air freshener compartment located at the front of the fan introduces a scent into an air flow created by the fan;
wherein the electronics module comprises a clock, an alarm, and a timer;
wherein the clock, the alarm, the timer, and motion of the plurality of fan blades is controlled by the remote control.

2. The fan with air freshening dispenser according to claim 1
wherein the air freshener compartment is a circular container at the front center of the blade cover;
wherein the air freshener compartment holds an air freshener;
wherein the air freshener compartment comprises a plurality of vents to allow the scent to escape from the air freshener compartment;
wherein the air freshener compartment opens to allow replacement or refilling of the air freshener.

3. The fan with air freshening dispenser according to claim 2 wherein the air freshener is a solid.

4. The fan with air freshening dispenser according to claim 2
wherein the electronics module comprises a housing, control circuitry, a sound transducer, and a display mounted to the front of the stand;
wherein the control circuitry comprises the clock, the alarm, and the timer;
wherein the clock tracks the time of day which the display shows on the display;
wherein the display is visible on the front side of the electronics module;
wherein the alarm is set to produce an alert noise at a specific time of day using the sound transducer;
wherein the timer is used to limit the duration of operation of the motor.

5. The fan with air freshening dispenser according to claim 4
wherein the electronics module further comprises a sensor for receiving a control signal sent by the remote control.

6. The fan with air freshening dispenser according to claim 5
wherein the remote control is a hand-held, battery powered device which controls the operation of the fan with air freshening dispenser from a distance;
wherein the remote control comprises a remote control enclosure, a plurality of controls, a transmitter, remote control circuitry, and one or more batteries;
wherein the remote control enclosure is a container which houses the remote control circuitry, the transmitter, the one or more batteries, and the plurality of controls;
wherein the plurality of controls is accessible for activation on the front side of the remote control enclosure;
wherein the remote control enclosure provides a battery door to allow access to the one or more batteries.

7. The fan with air freshening dispenser according to claim 6
wherein the plurality of controls activate features of the fan with air freshening dispenser;
wherein activating one of the plurality of controls causes the remote control circuitry to send the control signal from the transmitter to the sensor on
wherein the control signal is encoded or modulated to indicate which of the plurality of controls was activated.

8. The fan with air freshening dispenser according to claim 7
wherein the plurality of controls are pushbuttons.

9. The fan with air freshening dispenser according to claim 7
wherein the motor housing is a cowling to cover the motor;
wherein the motor housing prevents contact with moving parts or electrically energized circuits of the motor;
wherein the motor housing is pivotally coupled to the stand on each side of the motor housing to allow adjusting of the up/down angle of the fan.

10. The fan with air freshening dispenser according to claim 9
wherein the rear of the motor housing comprises a manual speed control;
wherein the manual speed control allows the fan to be turned on or off;
wherein the manual speed control allows selection of a fan speed by turning the manual speed control;
wherein the manual speed control provides an input signal to the control circuitry in the electronics module that allows the control circuitry to combine input from the manual speed control and input from the remote control into a final determination of the activation state and speed of the fan.

11. The fan with air freshening dispenser according to claim 9
wherein the motor is an electrical motor;
wherein the motor cause rotation of a motor shaft when the motor is electrically energized;
wherein the motor is electrically energized from a power source;
wherein the electronics module controls the electrical energy reaching the motor.

12. The fan with air freshening dispenser according to claim 11
wherein the motor shaft is coupled to a central hub;
wherein rotation of the motor shaft causes rotation of the plurality of fan blades via the central hub.

13. The fan with air freshening dispenser according to claim 12
wherein the motor causes oscillatory side-to-side motion of the fan relative to the stand through gearing and a crank mechanism.

14. The fan with air freshening dispenser according to claim 12
wherein the plurality of fan blades comprise 2 to 5 paddles that are coupled to the central hub and are inclined from being co-planar with each other;
wherein the plurality of fan blades are all inclined in the same direction and by the same angle of inclination with respect to the central hub;
wherein when the motor causes rotation of the central hub, the plurality of fan blades work co-operatively with each other to push air through the plurality of fan blades in the same direction.

15. The fan with air freshening dispenser according to claim 14
wherein the blade cover is a protective cover over the plurality of fan blades;
wherein the blade cover is a mesh or grill that allows the air flow through while preventing an object from touching the plurality of fan blades;
wherein the blade cover surrounds the plurality of fan blades and couples to the motor housing.

16. The fan with air freshening dispenser according to claim 15
wherein the stand is an upright support for the fan;
wherein the stand elevates the fan above a support surface or above the floor;
wherein the stand comprises an upright armature and a base;
wherein the base rests on the support surface to provide a stable platform for the fan with air freshening dispenser;
wherein the top of the base is coupled to the lower end of the upright armature;
wherein the upper end of the upright armature is pivotally coupled to the motor housing.

17. The fan with air freshening dispenser according to claim 16
wherein the electronics module is coupled to the front of the upright armature below the blade cover.

18. The fan with air freshening dispenser according to claim 17
wherein the upright armature comprises a height adjustment;
wherein the height adjustment allows an upper section of the upright armature to slide relative to a lower section of the upright armature when the height adjustment has been disengaged;
wherein the height adjustment allows the overall height of the fan above the support surface or above the floor to be changed;
wherein once the fan has been moved to a desired height, the height adjustment is engaged to hold that height.

19. The fan with air freshening dispenser according to claim 18
wherein the height adjustment comprises a twist-lock ring that locks or releases a telescopic joining of the upper section of the upright armature and the lower section of the upright armature.
